(19) United States
(12) Patent Application Publication
(10) Pub. No.: US 2019/0276828 A1
(43) Pub. Date: Sep. 12, 2019

(54) CONJUGATES FOR SENSING

(71) Applicant: Purdue Research Foundation, West Lafayette, IN (US)

(72) Inventors: ALEXANDER Wei, West Lafayette, IN (US); Chongli Yuan, West Lafayette, IN (US)

(73) Assignee: Purdue Research Foundation, West Lafayette, IN (US)

(21) Appl. No.: 16/345,046

(22) PCT Filed: Oct. 25, 2017
(86) PCT No.: PCT/US2017/058177

§ 371 Date: Apr. 25, 2019

Related U.S. Application Data
(60) Provisional application No. 62/412,301, filed on Oct. 25, 2016.

(51) Int. Cl. C12N 15/115; G01N 33/53

(52) U.S. Cl. CPC C12N 15/115; C12N 2310/16; G01N 33/5308

(57) ABSTRACT

The present disclosure relates to aptamer-binding partner conjugates for detection of an analyte in a sample; a substrate having attached thereto the aptamer-binding partner conjugate according to the invention; a method for the detection of an analyte in a sample comprising use of said aptamer-binding partner conjugate; and a binding partner for conjugating to an aptamer to generate an aptamer-binding partner conjugate.

### TECHNICAL FIELD

The present disclosure relates to aptamer-binding partner conjugates for detection of an analyte in a sample; a substrate having attached thereto the aptamer-binding partner conjugate according to the invention; a method for the detection of an analyte in a sample comprising use of said aptamer-binding partner conjugate; and a binding partner for conjugating to an aptamer to generate an aptamer-binding partner conjugate.

### BACKGROUND

Aptamers are synthetic receptors that can adopt local or global conformations that enable them to bind molecular analytes with high affinity. Aptamers are generally considered as biopolymers and can be made from natural or unnatural oligonucleotides, amino acids, or hybrid structures. A common feature or strategy in aptamer generation is the ability to identify high-affinity molecules within a large library of structures by “directed evolution,” in which candidate receptors are subjected to multiple cycles of chemical selection steps, followed by self-amplification and further screening. Aptamers with high specificity and nanomolar binding affinities for a target analyte have been obtained through this process.

Aptamers offer several advantages over antibodies, the current gold standard for receptor engineering. Aptamers are less massive (ca. 20 kDa vs. 160 kDa) and also less fragile than antibodies, and can be produced on demand by well-established synthetic methods. Antibodies must be produced through mammalian cultures which require a high overhead and a continuous investment in animal maintenance. Antibody-antigen recognition is not automatic: a great deal of art is needed to produce antibodies with specificity for a given analyte, often requiring several years for optimization. When such factors are combined, the development cost for customized antibodies can exceed that of aptamers by two orders of magnitude.

Methods for conjugating DNA or peptide oligomers onto gold nanoparticles (AuNPs) are well established, and can be achieved with high surface density (Sapsford 2013). In comparison, reliable methods for attaching oligomers onto preformed silver nanoparticles (AgNPs) are far less common. Recently, workers have reported that DNA oligomers with polycytidine sequences (5′-polyCₙ, n>20) form stable adsorbates on AgNP surfaces (Zhu 2015); other approaches involve DNA conjugated to thioalkyl or polysulfide linkers (Sapsford 2013). Further, DNA aptamers on AgNPs have been used for cell-surface recognition and the detection of protein analytes, based on Forster resonance energy transfer (FRET) and displacement of an antisense strand (Li 2013, Li 2015).

Dithiocarbamates (DTCs) are also excellent ligands for anchoring molecular ligands onto metal surfaces. They can be easily generated in situ by the addition of nucleophilic amines to Carbon disulphide (CS₂) under mildly basic conditions, and their chemisorption has been shown to be robust against desorption by competing adsorbates and at high temperatures (Zhao 2005, Wei 2010). DTC ligands are capable of chemisorption onto many types of inorganic substrates, including colloidal Ag, although their relative photostability needs to be further defined. We have determined that amine-terminated DNA oligomers are readily converted into DTCs at micromolar concentrations in aqueous buffer, and can be anchored onto AgNPs with high resistance to thermal desorption.

The vast majority of DNA and aptamer-based sensors involve either displacement of a dye-labelled strand by the analyte, and/or disruption of a FRET donor-acceptor pair upon analyte binding. There have also been attempts to use metal-enhanced fluorescence (MEF) as a turn-on mechanism for analyte recognition (Kang 2011).

We therefore describe herein a novel aptamer-binding partner conjugate for detection of an analyte in a sample that can be attached to an inorganic substrate, and further engineered to take advantage of MEF signal enhancement. The aptamer-binding partner conjugate would remain attached to the nanoparticle probe, with the fluorophore at a finite distance from the metal nanoparticle surface, for maximum MEF.

### STATEMENTS OF INVENTION

Accordingly, the invention provides an aptamer-binding partner conjugate for detection of an analyte in a sample wherein:

said aptamer comprises an analyte binding site and near or adjacent thereto said aptamer has a functionalised site for attaching same to a selected substrate; and

said binding partner comprises a signaling molecule including or attached to a linker, and integral with said linker or attached thereto, at a first end or part, is a sequence of base units complementary or substantially complementary to a sequence of base units in or adjacent said analyte binding site of said aptamer but whose binding affinity for said aptamer is such said sequence is displaceable by the analyte to be detected by said aptamer and, at a second remote end or part of said linker, is a sequence of base units complementary or substantially complementary to a sequence of base units in said aptamer remote from said analyte binding site; whereby

upon conjugation of said aptamer and said binding partner, said complementary sequences hybridise thus modulating the optical properties of said signaling molecule, with further modulation in the presence of said analyte.

In a preferred embodiment of the invention said aptamer comprises base units that are nucleotides, ideally, selected from the group comprising: ssDNA and ssRNA.

Moreover, said complementary sequence of base units preferably comprise biopolymers known to bind DNA or RNA such as, but not limited to, oligonucleotides, peptide nucleic acid, locked nucleic acid and oligodeoxynucleotides such as deoxyribonucleic acid or ribonucleic acid.

In a further preferred embodiment of the invention said functionalized site comprises a linker selected from the group comprising: a hydroxylamine, a hydrazide, a aminoalkoxy group, a polycytidine, a cytosine, a thioalkyl group, a polysulphide, a thiol, a dithiocarbamate, a carbodithioate, a dihydrolipoic acid, an isocyanide, a cyclam, a phosphine, a polyamine, an amine, a hydrazide, an aminooxy ligand, a thiolate and an alkoxide.

More preferably, said linker is designed to hold said signaling molecule, in the absence of said analyte and when attached to said substrate, within 1 nm, 2 nm, 3 nm, 4 nm, or 5 nm of said substrate. Additionally or alternatively, said binding partner is designed so that said analyte binding site thereof is located at or near to said functionalized site whereby said analyte binding site, in the absence of said analyte and when attached to said substrate, is within 1 nm, 2 nm, 3 nm, 4 nm, or 5 nm of said substrate.

Additionally, preferably, said binding partner is designed so that said sequence of base units in said aptamer remote from said analyte binding site, in the presence of said analyte and when attached to said substrate, is greater than 8 nm, 9 nm or 10 nm, 11 nm, 12 nm, 13 nm, 14 nm or 15 nm from said substrate.

In a further preferred embodiment said signaling molecule emits electromagnetic energy, ideally, in the visible spectrum. Most ideally, said signaling molecule emits a fluorescent signal. Ideally, said signaling molecule is a near-infrared fluorophore that emits in the range of between 450 and 650 nm. Alternatively, said signaling molecule emits in the range of between 650 and 900 nm.

Yet more preferably still, said signaling molecule has an absorption band that overlaps with the plasmon resonance of said substrate.

In a preferred embodiment, said substrate is made of, or coated with, silver, gold, aluminium, copper or platinum metals; or is made of, or coated with, at least one silver, gold, aluminium, copper or platinum colloid or an alloy thereof. Alternatively, said substrate is made of, or coated with, dielectric particles with a large absorption/scattering cross section for wavelengths of interest, such as, dyed-impregnated micro-silica particles.

Typically, but not exclusively, said substrate is a film or a nanoparticle, most typically a nanoparticle or a plurality thereof.

It will be apparent that said analyte binding site is specific for said analyte to be detected. Preferably, said analyte is any substrate the aptamer can recognise but most typically is selected from the group comprising: protein, enzyme, antigen, receptor, hormone, metabolite, an organic molecule and carbohydrate.

According to a further aspect of the invention there is provided a substrate having attached thereto the aptamer-binding partner conjugate according to the invention. Most ideally, the substrate is a plurality of nanoparticles.

According to a yet further aspect of the invention there is provided a method for the detection of an analyte in a sample comprising:
a) exposing said sample to a substrate according to the invention;
b) detecting the emittance of a signal from the said signaling molecule; and
c) where the emitted signal is either only present in the presence of said sample or where the emitted signal is enhanced or reduced in the presence of said sample concluding said analyte is present in said sample.

In a preferred method part b) further comprises measuring signal strength to provide a measure of the amount of analyte present in said sample.

Most typically part a) further involves analyte in said sample displacing said complementary or substantially complementary sequence of base units for said aptamer binding site in said binding partner and so allowing the aptamer to change shape whereby the signaling molecule is located greater than 8 nm, 9 nm or 10 nm, 11 nm, 12 nm, 13 nm, 14 nm or 15 nm from said substrate. In this embodiment of the method part b) further involves metal-enhanced fluorescence MEF and so detecting the emittance of a signal from the said signaling molecule involves measuring MEF.

Most ideally still, in the absence of said analyte to be detected, said signaling molecule is held within 1 nm, 2 nm, 3 nm, 4 nm or 5 nm of said substrate and so quenching of any emitted signal occurs whereby in the presence of analyte the relative size of the signal is enhanced. Ideally, said signal is enhanced at least tenfold.

According to a yet further aspect of the invention there is provided binding partner for an aptamer-binding partner conjugate comprising:
a) a signaling molecule including or attached to a linker,
b) integral with said linker or attached thereto, at a first end or part, is a sequence of base units complementary or substantially complementary to a sequence of base units in or adjacent an analyte binding site of said aptamer but whose binding affinity for said aptamer is such said sequence is displaceable by the analyte to be detected by said aptamer and,
c) at a second remote end or part of said linker, is a sequence of base units complementary or substantially complementary to a sequence of base units in said aptamer remote from said analyte binding site, and thus unaffected by analyte binding.

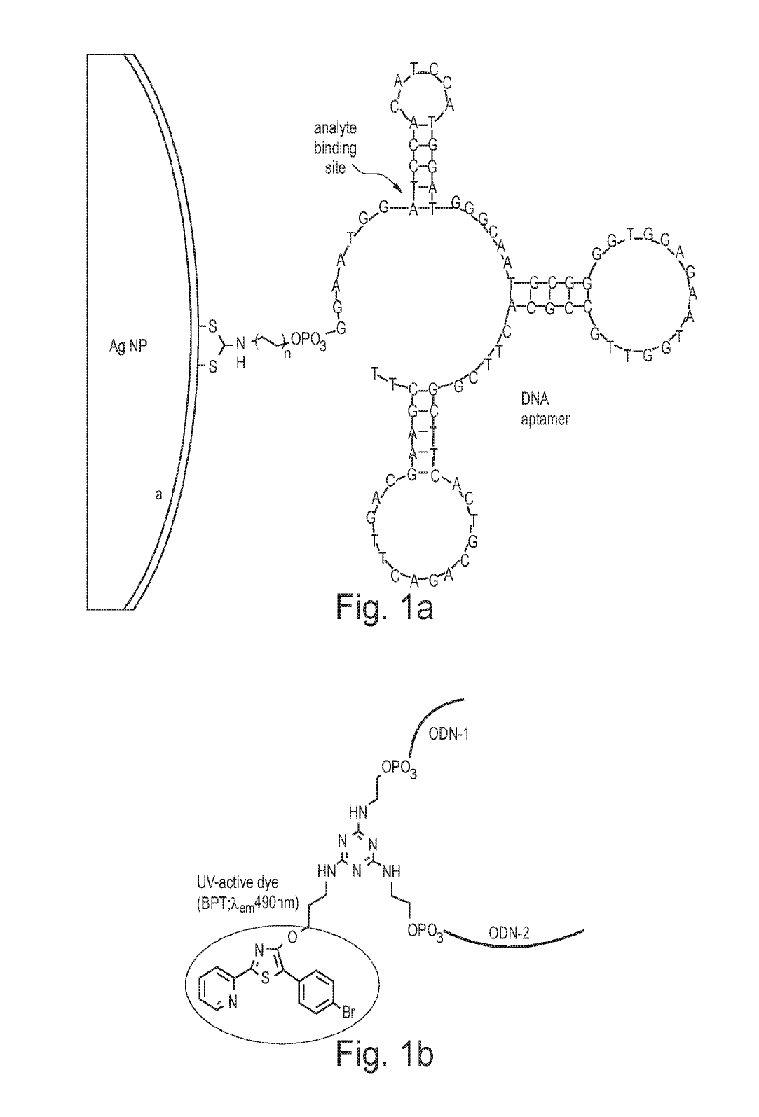

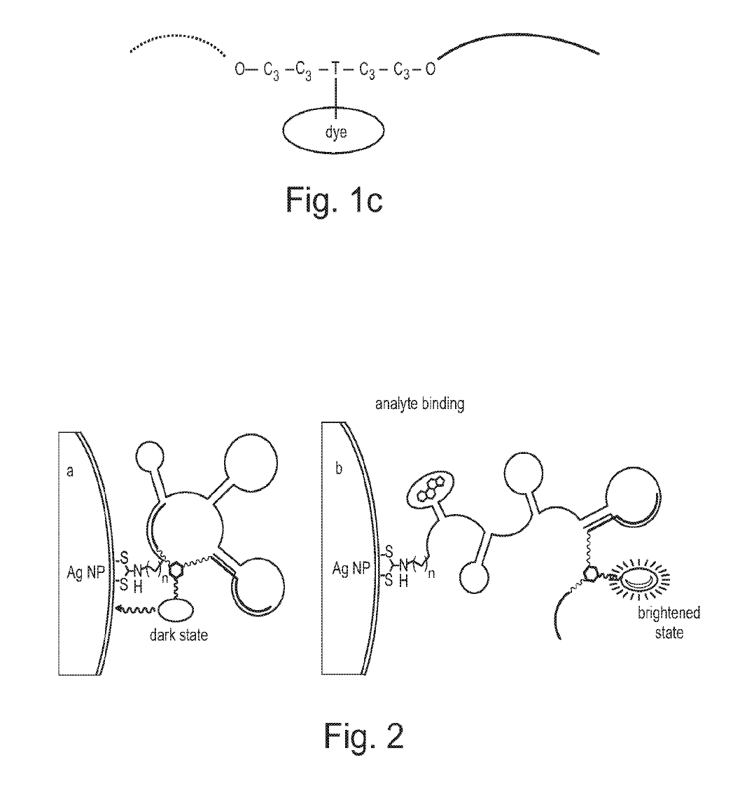

In this disclosure, we therefore describe an original system that in one particular embodiment combines (i) colloidal Ag nanoparticles, (ii) DNA aptamers with complementary oligomers, and (iii) fluorescent dyes with an absorption band that overlaps with the plasmon resonance of AgNPs. The latter feature permits the use of metal-enhanced fluorescence to maximize the emission of reporter dyes in response to analyte binding. Aptamers will be anchored at one end onto AgNPs with the analyte binding site close to the metal surface, preferably within 3 nm (FIG. 1a). The NP-bound aptamer will be hybridized with a molecular construct/binding partner comprised of a fluorescent reporter dye and two complementary DNA oligomers targeting two different sites in the aptamer, one relatively short segment (FIG. 1b, ODN-1) associated with the analyte binding region, and a relatively longer segment (FIG. 1b, ODN-2) distal from the surface anchor.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

An embodiment of the present invention will now be described by way of example only with reference to the following wherein:

FIG. 1a shows a DNA aptamer anchored at one end onto Ag nanoparticle, with an analyte binding site close to metal surface, 1b shows a binding partner/signaling complex comprised of two oligodeoxynucleotide (ODN) strands and a fluorescent reporter dye (p-(bromophenyl)pyridylthiazole (BPT)), and 1c shows an alternative binding partner/signaling complex to that shown in 1b, the complex comprising ODN1-C3 spacer-C3 spacer-Fluorescein dT-C3 spacer-C3 spacer-ODN2.

FIG. 2 shows (a) AgNP-aptamer hybridized with a binding partner/signaling complex, with quenching of dye emission by metal surface, and (b) that displacement of ODN-1 by analyte induces a large conformational change, disrupting the quenching effect. The dye emission can be further amplified by metal-enhanced fluorescence.

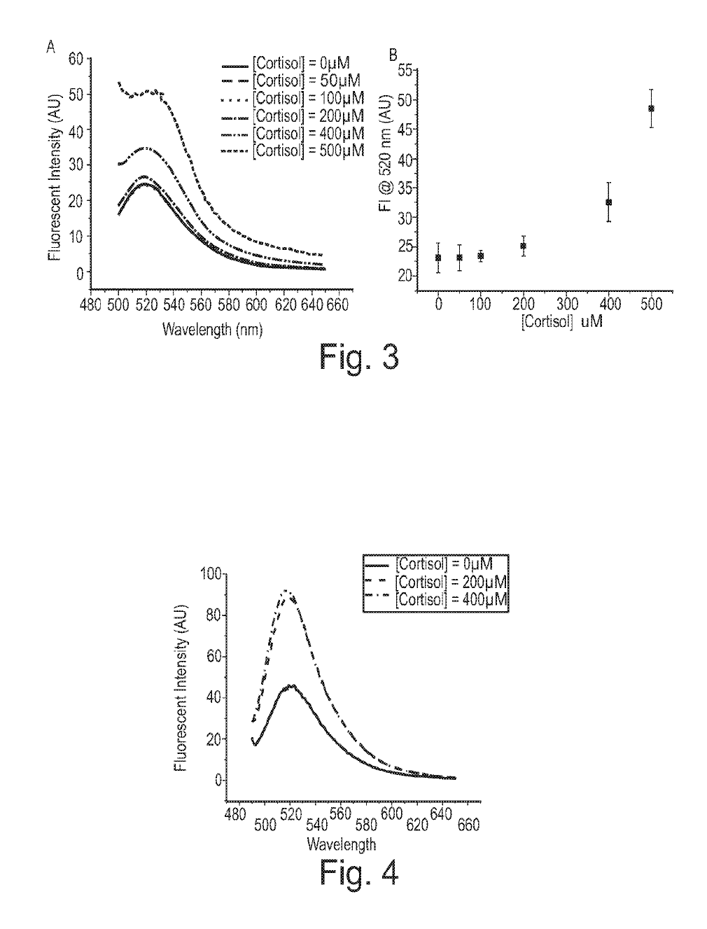

FIG. 3 shows (A) fluorescent intensity change of AgNP-CBA-signal complex conjugates, in response to cortisol (up to 500 μM). The AgNPs were treated with poly(deoxyinosinic-deoxycytidylic) acid (Poly(dI-dC)) prior to conjugation to prevent aggregation. 100-nm AgNPs were functionalized with cortisol-binding aptamer (CBA) having DTC anchor at the 5′-end. A complementary three-arm fragment (“conjugate”) with internal Fluorescein dT label as shown in FIG. 1c was used. In these experiments, samples were unwashed after treatment with conjugate; spectra have been corrected for background fluorescence. [CBA]=15 nM; [CBA: conjugate]=4:1; λₑₓ=460 nm; and B) peak fluorescence intensity of AgNP-CBA-signal complex conjugates as a function of cortisol concentration (N=2).

FIG. 4 shows AgNP-CBA-signal complex conjugates (15 nM) exposed to two different concentrations of cortisol (200 and 400 μM). The spectra of these mixtures show a two-fold increase in fluorescence intensity at 200 μM with no significant increase at 400 μM, suggesting signal saturation at the former level.

### DETAILED DESCRIPTION

Methods and Materials

Preparation of the Conjugates

The signaling dye p-(bromophenyl)pyridylthiazole (BPT) and its 4′-O-(3-azido)propyl derivative were prepared as previously described (Wolfram et al, Beilstein J. Org. Chem. 2014, 10, 2470-2479). 3-Azidopropyl BPT (105 mg, 0.25 mmol) and triphenylphosphine (66.7 mg, 0.25 mmol) were added to a microwave reaction tube and dissolved in 5 mL tetrahydrofuran, forming a clear, light yellow solution. This was heated for 10 min at 70° C. in a microwave reactor; concentration produced a yellow solid with 97% mass recovery, which was used without purification. 1H NMR spectroscopy revealed a doublet of triplets at 8.08 ppm, indicative of the desired aza-ylide, with a large ³J_{PH} splitting of 15 Hz. ¹H NMR (300 MHz, Chloroform-d) δ 8.57 (ddd, J=0.9, 1.7, 4.8 Hz, 1H), 8.08 (dt, J=7.9, 1.1 Hz, 1H), 7.79-7.24 (m, 30H), 4.63 (t, J=6.0 Hz, 2H), 3.34 (dt, J=15, 6.8 Hz, 2H), 2.12 (quint, J=6.1 Hz, 2H). Cyanuric chloride (1.6 equiv. relative to BPT aza-ylide) was dissolved in 0.25 mL of dichloromethane (DCM) and stirred at −20° C. for 15 minutes, then treated with 3-azidopropyl BPT (1.0 equiv.) dissolved in 0.75 mL of DCM, followed by neat trimethylamine (1.2-1.5 equivalents). The reaction mixture changed from a pale yellow to a bright yellow solution, and was allowed to warm to 10° C. over a period 2 hours before quenching with 5 mL of aqueous sodium bicarbonate and stirring for an additional 5 minutes, during which it turned a light yellow colour. The product was extracted with DCM (3×5 mL), dried over sodium sulfate, filtered and concentrated to a yellow solid. The mono-adduct is moisture-sensitive and found to be unstable over long periods of storage, and best used when freshly prepared. ¹H NMR (300 MHz, CDCl₃) δ 8.62 (ddd, J=0.9, 1.8, 4.8 Hz, 1H), 8.25 (dt, J=8.0, 1.1 Hz, 1H), 7.85 (dt, J=7.8, 1.7 Hz, 1H), 7.73-7.31 (m, 30H), 4.66 (t, 2H), 3.73 (q, J=6.3 Hz, 2H), 2.14 (quint, J=6.0 Hz, 2H).

The crude BPT-triazine conjugate above can be dissolved in acetonitrile, then treated with a nucleophilic amine (ODN1-NH₂) and triethylamine (1 equiv. each) and stirred for 12 hours at room temperature. The crude adduct can be concentrated to dryness, then redissolved in tetrahydrofuran and treated with a second nucleophilic amine (ODN2-NH₂) and triethylamine at reflux for several hours. The resulting signaling complex, which is diagrammatically represented in FIG. 1b, can be isolated by trituration and characterized by gel electrophoresis and MALDI-MS. The signaling complex comprises three distinct regions or arms, namely an ODN-1 arm, an ODN-2 arm and a UV-active dye arm.

An alternative signaling complex, which is diagrammatically represented in FIG. 1c, was prepared using conventional synthetic techniques. Like the BPT-triazine conjugate mentioned above, this signaling complex comprises three distinct regions, namely an ODN-1 arm, an ODN-2 arm and a fluorescent dye arm. The fluorescent dye arm, comprising C3 spacer-C3 spacer-Fluorescein dT-C3 spacer-C3 spacer, was obtained commercially from Integrated DNA Technologies, BVBA.

In situ dithiocarbamate formation was performed using 80 μM DNA with a 5′-hexylamine linker in deaerated 4:1 aqueous sodium borate buffer:methanol. The amine-functionalized DNA was treated with triethylamine and carbon disulphide diluted in methanol (final concentrations 5.7 and 2.5 mM respectively) and allowed to stand at room temperature for one hour. A 20% conversion was observed based on UV-vis spectra of the mPEG-DTC (ε ˜8000 M⁻¹ cm⁻¹), corresponding to 16 μM of DNA-DTC.

Conjugation of DNA-DTCs onto Poly(dI-dC) stabilized Ag NPs was performed by combining the solution above with a 1-mL suspension of said Ag NPs (dₙ=40 nm; O.D. 0.95 at 410 nm), followed by centrifugation at 4000 rpm for 30 min, then redispersed into DTC solutions by vortex mixing and allowed to sit for 1 hour at room temperature. The resultant DTC-stabilized NPs were centrifuged again and resuspended in the desired solution by vortex mixing and 30 seconds of sonication.

Use of the Aptamer-Binding Partner Conjugate for Detection of an Analyte in a Sample

Cortisol-binding Aptamer (‘CBA’) of a defined sequence feature was synthesized using a chemical synthesis approach. The CBA contained a carbodithioate anchor, and was physically adsorbed to the surface of AgNP. AgNP-CBA-signal complex conjugates were prepared according to the following procedure: AgNP adsorbed CBA (15 nM) was suspended in 15 mM borate buffer (pH 9.4) and mixed with aqueous solutions of the fluorescein dt-based signal complex (originally at 100 μM) at defined stochiometric ratios (typically 2:1 or 4:1). The mixtures were adjusted with 500 mM NaH₂PO₄ buffer (pH 7.9) to a final phosphate concentration of 25 mM, briefly vortexed then heated to 95° C. for 3 minutes, and cooled slowly to RT over 4-6 hrs. The sample was further cooled to 4° C. and protected from light for at least 12 hours prior to any measurements.

The assembled detection complex, i.e. AgNP-CBA-signal complex conjugate, was mixed with standard samples of known analyte (Cortisol) concentrations. Samples were examined using fluorescence measurements, carried out using a Cary Varian Eclipse fluorophotometer. The excitation wavelength was set at 460 nm and the emission was collected from 500 to 650 nm. For analyte titration experiments, aliquots of aqueous cortisol (800 μm) were mixed with the assembled detection complex using a micropipette for agitation. All samples were allowed to equilibrate for at least 5 minutes prior to fluorescence analysis. A standard curve correlating signal intensities with analyte concentrations can be established. This curve can be used as a calibration to enable direct conversion between signal strength and analyte concentration.

A sample with unknown analyte concentration (test sample) can be mixed with the assembled detection complex. The resulting signal can be compared against the calibration curve to enable the quantification of analyte concentrations.

Results

With reference to FIG. 1 it can be seen than a selected aptamer, i.e. specific for a particular analyte to be detected, is functionalised at a particular site i.e. near to said aptamer binding site, so that it can be attached to a preferred substrate, in this embodiment a silver coated nanoparticle. The coating can be an alloy or colloid coating, alternatively the nanoparticle may be made of silver.

Binding partners for the aptamer are shown in FIGS. 1b and 1c. They each comprise a signaling molecule, in this embodiment a fluorescent dye, which is attached to two oligonucleotides (ODN-1 and ODN-2), in this embodiment made from deoxynucleotides. A linker is used to attach the two oligonucleotides to the dye. The two oligonucleotides differ in that one (ODN-1) comprises a sequence of nucleotides that is complementary to, or at least sufficiently complementary to the aptamer binding site for the analyte to be detected so that it hybridises therewith; and the other (ODN-2) comprises a sequence of nucleotides that is complementary to, or at least sufficiently complementary to a sequence of base units in said aptamer remote from said analyte binding site such that it hybridises therewith. The two oligonucleotides are therefore of different sequence structure and possibly also different lengths.

Moreover the oligonucleotide that is complementary to the aptamer binding site has a binding affinity for said site such that in the presence of analyte to be detected it is displaced. Thus, with reference to FIG. 2 it can be seen that in the absence of analyte to be detected said oligonucleotide binds to said aptamer binding site and so said signaling molecule is held near to said aptamer binding site. Given the aptamer is functionalised for attaching to said substrate near its binding site, the signaling molecule is thus held near to said substrate/nanoparticle and so quenching of signal from said dye takes place, essentially switching off/dampening the signal from the signaling molecule.

Conversely, in the presence of analyte to be detected, the oligonucleotide that is complementary to the aptamer binding site is displaced and the binding partner undergoes a conformational change, essentially untethering the dye from its position near the nanoparticle surface and enabling it to move to a distant location, ideally 8-15 nm from the substrate surface where maximum MEF can be achieved, thus enhancing the signal from the dye and essentially switching on the signal from the system. MEF can amplify quantum yield by an order of magnitude or more and so this feature is preferred.

Notably, the dye-labelled binding partner remains attached to the aptamer due to the presence of the other oligonucleotide that binds the aptamer remote from said analyte binding site. Ideally this oligonucleotide has a high degree of complementarity with said aptamer and/or a longer sequence (with respect to said other oligonucleotide) whereby it securely anchors the binding partner to the aptamer. Thus the positioning of the dye in the presence of the analyte to be detected is a function of the size/shape of the aptamer and/or the binding partner. This feature is therefore considered when designing a binding partner for an aptamer. The oligonucleotide of the binding partner that binds the aptamer remote from said analyte binding site is designed to bind the aptamer within a region that is 8-15 nm from the substrate surface (having regard to the length of anchoring molecule used to attach the aptamer to the substrate and the shape of the aptamer in the presence of analyte).

The analyte-detection performance of such AgNP-CBA-signal complex conjugates has been illustrated using a fluorescence titration assay, which was developed to illustrate the turn-on fluorescence response of such conjugate structures in the presence of cortisol. The experimental results, which are shown in FIG. 3, clearly indicate that an increase in peak fluorescence intensity was observed as a function of cortisol concentration.

The titration experiment referred to above is subject to hysteresis effects, which complicate the quantitative analysis of the observed fluorescence response to the presence of analyte. The study was, therefore, repeated using single-point measurements, the results of which are shown in FIG. 4, which produced changes as step functions of cortisol exposure.

We herein described a system for the detection of a selected analyte in a sample which is highly specific for the analyte to be detected and employs the use of affordable and available aptamer technology and a reliable and enhanced signaling mechanism. In particular, the experiments described above demonstrate that the level of fluorescence from said system increases as analyte is added. Consequently, the claimed aptamer-binding partner conjugates are suitable for use in the quantitative detection of analytes.

### REFERENCES

Sapsford, K. E.; Algar, W. R.; Berti, L.; Gemmill, K. B.; Casey, B. J.; Oh, E.; Stewart, M. H.; Medintz, I. L., Functionalizing Nanoparticles with Biological Molecules: Developing Chemistries that Facilitate Nanotechnology. Chem. Rev. 2013, 113, 1904-2074.
Zhu, D.; Chao, J.; Pei, H.; Zuo, X.; Huang, Q.; Wang, L.; Huang, W.; Fan, C., Coordination-Mediated Programmable Assembly of Unmodified Oligonucleotides on Plasmonic Silver Nanoparticles. ACS Appl. Mater. Interfaces 2015, 7, 11047-11052.
Li, H.; Wang, M.; Wang, C.; Li, W.; Qiang, W.; Xu, D., Silver Nanoparticle-Enhanced Fluorescence Resonance Energy Transfer Sensor for Human Platelet-Derived Growth Factor-BB Detection. Anal. Chem. 2013, 85, 4492-4499.
Li, H.; Hu, H.; Zhao, Y.; Chen, X.; Li, W.; Qiang, W.; Xu, D., Multifunctional Aptamer-Silver Conjugates as Theragnostic Agents for Specific Cancer Cell Therapy and Fluorescence-Enhanced Cell Imaging. Anal. Chem. 2015, 87, 3736-3745.
Zhao, Y.; Perez-Segarra, W.; Shi, Q.; Wei, A., Dithiocarbamate Assembly on Gold. J. Am. Chem. Soc. 2005, 127, 7328-7329.
Wei, A.; Zhao, Y. Preparation of Dithiocarbamate Ligands for Nanotechnology and Biosensing Applications.” U.S. Pat. No. 7,803,568 (application filed Apr. 20, 2006; issued Sep. 28, 2010).
Kang, K. A.; Wang, J.; Jasinski, J. B.; Achilefu, S., Fluorescence Manipulation by Gold Nanoparticles: From Complete Quenching to Extensive Enhancement. J. Nanobiotechnol. 2011, 9, 1-13.

1. An aptamer-binding partner conjugate for detection of an analyte in a sample wherein:
said aptamer comprises an analyte binding site and near or adjacent thereto said aptamer has a functionalised site for attaching same to a selected substrate; and
said binding partner comprises a signaling molecule including or attached to a linker, and integral with said linker or attached thereto, at a first end or part, is a sequence of base units complementary or substantially complementary to a sequence of base units in or adjacent said analyte binding site of said aptamer but whose binding affinity is such said sequence is displaceable by the analyte to be detected by said aptamer and, at a second remote end or part of said linker, is a sequence of base units complementary or substantially complementary to a sequence of base units in said aptamer remote from said analyte binding site; whereby
upon conjugation of said aptamer and said binding partner, said complementary sequences hybridise thus equipping said aptamer with a signaling molecule.

2. The aptamer-binding partner conjugate according to claim 1 wherein said aptamer comprises base units that are nucleotides.

3. The aptamer-binding partner conjugate according to claim 2 wherein said aptamer has base units that are selected from the group comprising: ssDNA and ssRNA.

4. The aptamer-binding partner conjugate according to claim 1 wherein said complementary sequence of base units comprise biopolymers known to bind DNA.

5. The aptamer-binding partner conjugate according to claim 4 wherein said biopolymers comprises oligonucleotides, peptide nucleic acid, locked nucleic acid and oligodeoxynucleotides.

6. The aptamer-binding partner conjugate according to claim 1 wherein said functionalized site comprises a linker selected from the group comprising: a hydroxylamine, a hydrazide, a aminoalkoxy group, a polycytidine, a cytosine, a thioalkyl group, a polysulphide, a thiol, a dithiocarbamate, a carbodithioate, a dihydrolipoic acid, an isocyanide, a cyclam, a phosphine, a polyamine, an amine, a hydrazide, an aminooxy ligand, a thiolate and an alkoxide.

7. The aptamer-binding partner conjugate according to claim 1 wherein said functionalized site comprises a linker that is designed to hold said signaling molecule, in the absence of said analyte and when attached to said substrate, within 1 nm, 2 nm, 3 nm, 4 nm or 5 nm of said substrate.

8. The aptamer-binding partner conjugate according to claim 1 wherein said binding partner is designed so that said analyte binding site is located at or near to said functionalized site whereby said analyte binding site, in the absence of said analyte and when attached to said substrate, is within 1 nm, 2 nm, 3 nm, 4 nm or 5 nm of said substrate.

9. The aptamer-binding partner conjugate according to claim 1 wherein said binding partner is designed so that said sequence of base units in said aptamer remote from said analyte binding site, in the presence of said analyte and when attached to said substrate, is greater than 8 nm, 9 nm or 10 nm, 11 nm, 12 nm, 13 nm, 14 nm or 15 nm from said substrate.

10. The aptamer-binding partner conjugate according to claim 1 wherein said signaling molecule emits electromagnetic energy.

11. The aptamer-binding partner conjugate according to claim 10 wherein said signaling molecule emits energy in the visible spectrum.

12. The aptamer-binding partner conjugate according to claim 11 wherein said signaling molecule emits a fluorescent signal.

13. The aptamer-binding partner conjugate according to claim 1 wherein said signaling molecule is a near-infrared fluorophore that emits in the range of between 650 and 900 nm.

14. The aptamer-binding partner conjugate according to claim 1 wherein said signaling molecule emits in the range of between 450 and 650 nm.

15. The aptamer-binding partner conjugate according to claim 1 wherein said signaling molecule has an absorption band that overlaps with the plasmon resonance of said substrate.

16. The aptamer-binding partner conjugate according to claim 1 wherein said substrate is made of, or coated with, silver, gold, aluminium, copper or platinum metals; or is made of, or coated with, at least one silver, gold, aluminium, copper or platinum colloid or an alloy thereof.

17. The aptamer-binding partner conjugate according to claim 1 wherein said substrate is made of, or coated with, dielectric particles with a large absorption/scattering cross section for wavelengths of interest, such as, dyed-impregnated micro-silica particles.

18. The aptamer-binding partner conjugate according to claim 16 wherein said substrate is a nanoparticle.

19. The aptamer-binding partner conjugate according to claim 1 wherein said analyte binding site is specific for said analyte to be detected.

20. The aptamer-binding partner conjugate according to claim 1 wherein said analyte is selected from the group comprising: a protein, an enzyme, an antigen, a receptor, a hormone, a metabolite, an organic molecule and a carbohydrate.

21-32. (canceled)
